(19) United States
(12) Patent Application Publication
(10) Pub. No.: US 2014/0271354 A1
(43) Pub. Date: Sep. 18, 2014

(54) METHODS AND SOLUTIONS FOR KILLING OR DEACTIVATING BACTERIA

(71) Applicants: Tsung-Chan Tsai, Cuyahoga Falls, OH (US); Sameer Kalghatgi, Fairlawn, OH (US); Daphne Pappas Antonakas, Hudson, OH (US); Robert L. Gray, Hudson, OH (US)

(72) Inventors: Tsung-Chan Tsai, Cuyahoga Falls, OH (US); Sameer Kalghatgi, Fairlawn, OH (US); Daphne Pappas Antonakas, Hudson, OH (US); Robert L. Gray, Hudson, OH (US)

(73) Assignee: EP Technologies LLC, Akron, OH (US)

(21) Appl. No.: 13/838,418

(22) Filed: Mar. 15, 2013

(51) Int. Cl. A61L 2/18

(52) U.S. Cl. CPC A61L 2/18
USPC 422/29

(57) ABSTRACT

Exemplary methods for killing or deactivating bacteria are provided herein. One exemplary method includes providing a plasma system and a filter below the plasma system. Energizing the plasma generator to create indirect plasma downstream of the filter and activating a fluid with the indirect plasma and applying the activated fluid to bacteria is shown to deactivate and kill bacteria.

### TECHNICAL FIELD

The present invention relates generally to methods and solutions for killing or deactivating bacteria.

### BACKGROUND OF THE INVENTION

Bacteria, such as, for example Escherichia coli (“E. coli”), cause thousands of illnesses every year. Current methods of killing or deactivating such bacteria include applying heat and UV radiation and washing with an alcohol based sanitizer, bleach, hydrogen peroxide, and an anti-bacteria soap.

### SUMMARY

Exemplary methods for killing or deactivating bacteria are provided herein. One exemplary method includes providing a plasma generator and a filter below the plasma generator. Energizing the plasma generator to create indirect plasma downstream of the filter and activating a fluid with the indirect plasma and applying the activated fluid to bacteria.

Another exemplary method of killing or deactivating bacteria includes activating a fluid with indirect plasma; and applying the fluid to a surface having bacteria on it.

An exemplary method of killing or deactivating E. coli is also provided. The method includes activating a fluid using indirect plasma and applying the fluid to E. coli bacteria.

In addition, exemplary solutions for killing or deactivating bacteria are also provided. One exemplary solution includes fluid activated by indirect plasma.

### BRIEF DESCRIPTION OF THE DRAWINGS

These and other features and advantages of the present invention will become better understood with regard to the following description and accompanying drawings in which:

FIG. 1 illustrates a prior art embodiment for creating activated water using direct plasma;

FIG. 2 illustrates a prior art embodiment for creating activated water using indirect plasma;

FIG. 3 illustrates an exemplary mesh filter for use in the exemplary embodiment of FIG. 2; and

FIG. 4 illustrates an exemplary embodiment of a methodology for killing or deactivating bacteria.

### DETAILED DESCRIPTION

Plasmas, or ionized gases, have one or more free electrons that are not bound to an atom or molecule. Plasmas may be generated using a variety of gases including, air, nitrogen, noble gases (He, Ar, Xe, Kr, etc), oxygen, carbon dioxide and mixtures thereof under an electric field. In addition, non-thermal plasmas provide high concentrations of energetic and chemically active species. They can operate far from thermodynamic equilibrium with high concentrations of active species and yet remain at a temperature that is substantially the same as room temperature. The energy from the free electrons may be transferred to additional plasma components creating additional ionization, excitation and/or dissociation. Fluid that is contacted with plasma becomes “activated” and is referred to herein as plasma activated fluid, and in some embodiments, the plasma activated fluid is plasma activated water.

In some embodiments, plasmas may contain superoxide anions [O₂.⁻], which react with H⁺ in acidic media to form hydroperoxy radicals, HOO.:[O₂.⁻]+[H⁺]→[HOO.]. Other radical species may include OH. and NO. in aqueous phase or the presence of air or gas. Treating water with plasma results in plasma activated water that may contain concentrations of one or more of ozone, H₂O₂, nitrates, nitrites, radicals and other active species.

Activating water with plasma to obtain plasma activated water is shown and described in co-pending U.S. Provisional Application Ser. No. 61/621,078 titled Sanitization Station Using Plasma Activated Fluid, filed on Apr. 6, 2012 and co-pending U.S. Provisional Application Ser. No. 61/710,263 titled Solutions and Methods of Making Solutions to Kill or Deactivate Spores Microorganisms, Bacteria and Fungus, filed on Oct. 5, 2012. Both of which are incorporated by reference herein in their entirety. Several other patents and applications such as: PCT Application Nos. WO 02/059046, titled Method of Activation of Chemically Pure and Potable Water and filed on Jan. 25, 2002; WO 2007/048806, titled Method for the Preparation of Biocidal Activated Water Solutions and filed Oct. 25, 2006; WO 2012/018891, which is titled Materials for Disinfection Produced by Non-Thermal Plasma and was filed on Aug. 3, 2011; and U.S. Pat. No. 7,291,314, titled Activated Water Apparatus and Methods and filed Dec. 20, 2001, are incorporated herein by reference in their entirety for their disclosure on activating fluid.

It is known to treat water or fluid with plasma to “activate” the water or fluid. One method of activating water is illustrated in FIG. 1, which is a prior art dielectric barrier discharge (“DBD”) plasma generating system 100. The prior art plasma generating system 100 includes a high voltage source 102, a conductor 104, a housing 108, a high voltage electrode 106 and a dielectric barrier 110. The plasma generating system 100 also includes a container 120 which is grounded with grounding conductor 122. During operation, the high voltage source 102 is turned on and plasma 130 forms below the dielectric barrier 110. High voltage power source 102 may be a DC power source, a high frequency AC power source, an RF power source, a pulsed DC power source, a pulsed AC power source, a microwave power source or the like. The power supply can be pulsed with a duty cycle of 0-100% and pulse duration of 1 nanosecond up to 1 microsecond.

The plasma contacts the water or fluid 126 and activates the water or fluid 126. Fluid 126 activated by direct contact with plasma is referred to herein as “direct plasma activated fluid.”

FIG. 2 illustrates an exemplary prior art system 200 for activating a fluid using indirect plasma. System 200 includes a high voltage power source 202. High voltage power source 202 may be a DC power source, a high frequency AC power source, an RF power source, a pulsed DC power source, a pulsed AC power source, a microwave power source or the like. The power supply can be pulsed with a duty cycle of 0-100% and pulse duration of 1 nanosecond up to 1 microsecond.

The exemplary system 200 includes a DBD plasma housing 208 connected to high voltage power source 202 by cable 204. Indirect barrier discharge plasma system 200 includes a high voltage electrode 206 and a dielectric barrier 210 located between high voltage electrode 206 and the fluid 226 that is to be activated. The indirect plasma generating system 200 also includes a fluid container 220. A filter 250 is also included. Filter 250 is a conductive mesh that is grounded by grounding conductor 222.

During operation of system 200, when high voltage electrode 206 is energized, plasma 230 forms below the dielectric barrier 210, and the filter 250 (if the filter 250 is made of a conductive material and grounded) prevents charged ions and electrons from passing through and contacting the fluid 226 to be activated. Thus, only neutral species pass through and activate the fluid 226. This is typically referred to as “afterglow” or “indirect” plasma. In some embodiments, the fluid is water. Fluid 226 activated by afterglow that passes through, or is created through filter 250, is referred to “indirect plasma activated fluid.”

The experimental data provided below generated by indirect plasma, utilized a copper mesh as a filter. FIG. 3 illustrates the exemplary copper mesh 300 that was utilized as filter 250. The copper mesh was a copper woven wire cloth having a 16×16 mesh size with a 0.011″ wire diameter and a 0.052″ opening size (67% opening area). A mesh made of different conducting materials, wire diameters and opening sizes may be used.

In the exemplary embodiments disclosed herein the fluid may be water. In some embodiments, the properties of the fluid may be altered prior to activation by plasma or indirect plasma to increase or decrease concentration of species, radicals and the like. For example, the pH of water may be adjusted to be acidic or basic. The pH may be adjusted by, for example, adding acid to the water prior to activation. The pH level may be lowered through the activation process. In one embodiment, the pH level of the activated water is about 2.0, in another the pH is between about 2.0 and 3.5, and in yet another is about 2.7. Still in another the pH is less than about 3.0, and in another embodiment is less than about 2.0. In one embodiment, the pH is about 2.0.

In addition, the properties of the activated fluid may be adjusted during the activation process itself by altering the gas that is ionized at the plasma area. For example, the gas that is ionized may be normal air, N₂, O₂, He, Ar, Xe, Kr, combinations thereof at various ratios, or the like. In some embodiments, one or more gases are used in the plasma generating process. In some embodiments, one or more noble gases are used in the plasma generating process, and in some embodiments, combinations of noble and other gases are used in the plasma generating process.

Further, additives may be added before or after the fluid is activated to increase efficacy or stabilization of the resulting solution. Other additives that may be used depending on the desired results include, for example, alcohol, silver salts, e.g., silver nitrate or silver chloride, or colloidal silver; zinc salts, e.g. zinc chloride, zinc lactate, or zinc oxide; suspensions containing metal nanoparticles; chlorhexidine; anionic, cationic, non-ionic and/or amphoteric surfactants; emulsifiers; hydrotropes; glycerol; chelating agents; alcohols; quaternary ammonium compounds, acids (organic or inorganic); bases; or surface tension decreasing agents.

Fluid 226 may be a source of water, or of water with additional additives. In one embodiment, the fluid is tap water, however, the water may be distilled water, deionized water, tap water, filtered water, saline, water with acidic properties, water with basic properties or water mixed with additives such as, for example, alcohol. In addition, other additives may be used to optimize generation or increase performance and/or increase stability. These additives may include, for example chelators to reduce metal degradation; surfactants to improve penetration of the solution to reduce the impact of organic load and/or buffers to adjust the pH. In addition, in some embodiments corrosion inhibitors may be added, such as, for example, inorganic sulfates, inorganic phosphates. In some embodiments, a zeolite buffering system may be used. In some embodiments, one or more of these additives are added prior to activation of the water.

In some embodiments, the prior art filter 250 may be replaced by a filter having a carulite catalyst to filter out ozone. Other materials and/or coatings may be used to block certain species in the plasma from passing through to the fluid. In some embodiments, multiple filters are utilized, thus a copper filter could be used to filter out charged elements, and a second carulite coated mesh could be used to filter out ozone. In addition, a wire mesh may be used for electromagnetic shielding. In some embodiments, the filter is conductive and is used to tune the electric field between the plasma generator and conductive filter to control the density and/or concentrations of reactive species that pass through the filter.

FIG. 4 illustrates an exemplary methodology 400 of killing or deactivating bacteria. The exemplary methodology begins at block 402. At block 404, fluid is placed in contact with indirect plasma to activate the fluid. In some embodiments, the fluid is activated for about 5 minutes or less. In some embodiments, fluid is activated for about 3 minutes or less. The bacterial is inactivated at block 406 and the methodology ends at block 408.

As described above, the fluid may be water, additives may be added to the fluid prior to activation with indirect plasma or after activation with indirect plasma.

Treating Escherichia coli (“E. coli”) bacteria with a fluid that contained indirect plasma activated water resulted in a fluid having superior kill power over fluid activated with direct plasma.

The below experiments were conducted on E. coli in solution. The plasma setup was a dielectric barrier discharge plasma system. An alternating voltage pulsed power supply was used in the experiment to generate plasma. The pulse frequency was 3.5 kHz and the pulse duration was 10 μs. The amplitude of the voltage pulse was 20 kV peak to peak with a 5 V/ns rise time. The gap distance between the plasma generating system and the treated surface was about 1 to 2 mm. The experiments used air as the plasma working gas under the pressure of 1 atmosphere (ambient pressure).

Activated fluid may be fluid activated by direct plasma or indirect plasma, also known as “afterglow.” Direct plasma is generated as described above. Indirect plasma is obtained by generating plasma in the presence of a grounded filter, such as, for example, a copper mesh. In one embodiment, the copper mesh is located proximate to the dielectric barrier of the DBD plasma system. The grounded copper mesh prevents the charged ions and electrons from passing through, but allows the neutral species to pass through and activate the fluid. Thus, the activated fluid or activated water may be activated by plasma or by indirect plasma. All of the embodiments described with reference to FIGS. 1-4 may be direct plasma or indirect plasma.

The generated plasma was applied directly to the tap water for the direct plasma, and for indirect plasma, the afterglow was applied to the water. The reactive species generated from the air plasma diffused in to and reacted with the water, further “activating” the treated water. The direct plasma activated water or indirect plasma activated water was then added to the solution containing E. coli.

For the E. coli inactivation tests, the standard testing method, ASTM 2315, was utilized. 10⁸ CFU/ml E. coli suspension was prepared in Physiological Saline (8.5 g/L NaCl). 10 μL of the E. coli bacteria solution was drawn and added to 990 μl of the plasma activated water. After being vortexed for 30 seconds, 0.1 ml of the mixture of the E. coli solution and the plasma activated water was added to 9.9 ml of neutralizer. The neutralizer solution containing E. coli bacteria was then diluted and plated on Tryptic Soy Agar. 24-hr incubation was performed at 37° C., followed by the estimation of colony forming units (CFU).

The chart below indicates results for direct plasma. 2.0 ml of tap water was activated by the direct plasma. 990 μl of the direct plasma activated water was mixed with 10 μl of the E. coli bacteria solution. And then the testing procedure described above was used to obtain the CFU of E. coli after the treatment using the direct plasma activated water. The test results demonstrated that treating E. coli for 30 seconds with direct plasma activated water (water exposed to plasma for 3 minutes) resulted in a 0.77 log reduction of the colony forming units per milliliter “CFU/ml” of bacteria. Treating E. coli for 30 seconds with plasma activated water (water exposed to plasma for 5 minutes) alone resulted in a 0.84 log reduction (CFU/ml) of bacteria.

| | Direct Plasma | Treatment | Log Reduction |
|---|---|---|---|
| Solution | Activation Time | Time | (CFU/ml) |
| 2.0 ml water | 3 min | 30 sec | 0.77 |
| 2.0 ml water | 5 min | 30 sec | 0.84 |

The chart below indicates results for indirect plasma. 2.0 ml of tap water was activated by the direct plasma. 990 μl of the direct plasma activated water was mixed with 10 μl of the E. coli bacteria solution And then the testing procedure described above was used to obtain the CFU of E. coli after the treatment using the direct plasma activated water. The test results demonstrated that treating E. coli for 30 seconds with indirect plasma activated water (water exposed to indirect plasma for 3 minutes) alone resulted in log reductions colony forming units per milliliter “CFU/ml” of bacteria of between 1.01 and 1.43. Treating E. coli for 30 seconds with indirect plasma activated water (water exposed to indirect plasma for 5 minutes) alone resulted in a 2.59 log reduction (CFU/ml) of bacteria.

| | Indirect Plasma | Treatment | Log Reduction |
|---|---|---|---|
| Solution | Activation Time | Time | (CFU/ml) |
| 2.0 ml of tap water | 3 min | 30 sec | 1.01, 1.43 |
| 2.0 ml of tap water | 5 min | 30 sec | 2.59 |

Thus, the experimental results demonstrate that indirect plasma activated water has a superior kill or deactivation of E. coli than direct plasma activated water.

While the present invention has been illustrated by the description of embodiments thereof and while the embodiments have been described in considerable detail, it is not the intention of the applicants to restrict or in any way limit the scope of the appended claims to such detail. Additional advantages and modifications will readily appear to those skilled in the art. Moreover, elements described with one embodiment may be readily adapted for use with other embodiments. Therefore, the invention, in its broader aspects, is not limited to the specific details, the representative apparatus and/or illustrative examples shown and described. Accordingly, departures may be made from such details without departing from the spirit or scope of the applicants' general inventive concept.

We claim:

1. A method for killing or deactivating bacteria comprising:
providing a plasma system;
providing a filter below the plasma system;
energizing the plasma system to create indirect plasma downstream of the filter;
activating a fluid with the indirect plasma; and
applying the activated fluid to bacteria.

2. The method of claim 1 wherein the bacteria comprises E. coli.

3. The method of claim 1 wherein the fluid comprises water.

4. The method of claim 3 further comprising an additive added to the water prior to activation.

5. The method of claim 3 further comprising an additive added to the water after activation.

6. The method of claim 4 wherein the plasma system is a dielectric barrier discharge plasma system.

7. The method of claim 1 wherein the fluid is activated for less than about 5 minutes.

8. The method of claim 1 wherein the fluid is activated for less than about 3 minutes.

9. A method of killing or deactivating bacteria comprising:
activating a fluid with indirect plasma; and
applying the fluid to a surface having bacteria on it.

10. The method of claim 9 wherein the bacteria comprises E. coli.

11. The method of claim 9 wherein the fluid comprises water.

12. The method of claim 11 further comprising an additive added to the water prior to activation.

13. The method of claim 11 further comprising an additive added to the water after activation.

14. The method of claim 9 wherein the plasma generator is a dielectric barrier discharge plasma system.

15. The method of claim 9 wherein the fluid is activated for less than about 5 minutes.

16. The method of claim 9 wherein the fluid is activated for less than about 3 minutes.

17. A method of killing or deactivating E. coli comprising:
activating a fluid using indirect plasma;
applying the fluid to E. coli bacteria.

18. The method of claim 17 wherein the fluid comprises water.

19. The method of claim 18 wherein the water is activated for less than about 3 minutes.

20. The method of claim 18 wherein the water is activated for less than about 5 minutes.
